(19) United States
(12) Patent Application Publication
(10) Pub. No.: US 2009/0281480 A1
(43) Pub. Date: Nov. 12, 2009

(54) OXYGENATION OF AQUEOUS SYSTEMS

(76) Inventors: David Orlebeke, McMinnville, OR (US); Joyce K. Orlebeke, Mcminnville, OR (US)

Correspondence Address: KOLISCH HARTWELL, P.C., 200 PACIFIC BUILDING, 520 SW YAMHILL STREET, PORTLAND, OR, 97204, US

(21) Appl. No.: 11/917,746

(22) PCT Filed: Jul. 5, 2006
(86) PCT No.: PCT/US06/26260

§ 371 Date: Dec. 14, 2007

Related U.S. Application Data
(60) Provisional application No. 60/696,906, filed on Jul. 5, 2005.

(51) Int. Cl. A61M 37/00; C02F 1/461; C02F 1/463

(52) U.S. Cl. 604/24; 205/756; 205/752

(57) ABSTRACT

Methods for electrolytic oxygenation of aqueous systems, including substantially immersing an anode and a cathode in an aqueous medium, injecting oxygen into the aqueous medium, and applying a current to the electrodes.

### TECHNICAL FIELD

The present invention relates generally to oxygenation of aqueous systems, and more particularly, to the oxygenation of aqueous systems in combination with electrolytic treatment.

### BACKGROUND

Electrolysis is typically defined as a process whereby an electric current is passed through an electrolytic solution or other appropriate medium, and a chemical reaction or physical process is enabled thereby. U.S. Pat. No. 6,802,956 (hereby incorporated by reference) describes electrolytic processes for treating wastewater and efficiently removing pollutants.

The presence of oxygen can be useful or even necessary in a variety of applications. In aquaculture, for example, the amount of oxygen present in the aquaculture medium may have a direct impact on the health of the cultivated species, as well as the maximum number of cultivated individuals that may be supported by a given volume of the medium. Oxygen may be added to wastewater in order to aid in wastewater treatment and/or pollutant removal. Additionally, oxygen therapy has been used to treat a variety of conditions and symptoms, for example where treatment includes residence in a hyperbaric chamber.

However, the addition of oxygen to aqueous media can require expensive and/or complex equipment. In particular, it can be difficult to increase oxygen concentrations to a level above the saturation point of the medium.

The addition of oxygen to aqueous systems using electrolytic process has been found to facilitate supersaturation of the aqueous media, and lends itself to a variety of applications.

### BRIEF DESCRIPTION OF THE DRAWINGS

FIG. 1 is a flowchart depicting a method of electrolytic oxygenation of aqueous media according to an aspect of the present invention.

FIG. 2 is a schematic representation, viewed from above, of an electrolytic cell according to an aspect of the present invention.

FIG. 3 is a schematic representation of an electrolytic aquaculture medium treatment system according to an aspect of the present invention.

### DETAILED DESCRIPTION OF PREFERRED EMBODIMENTS

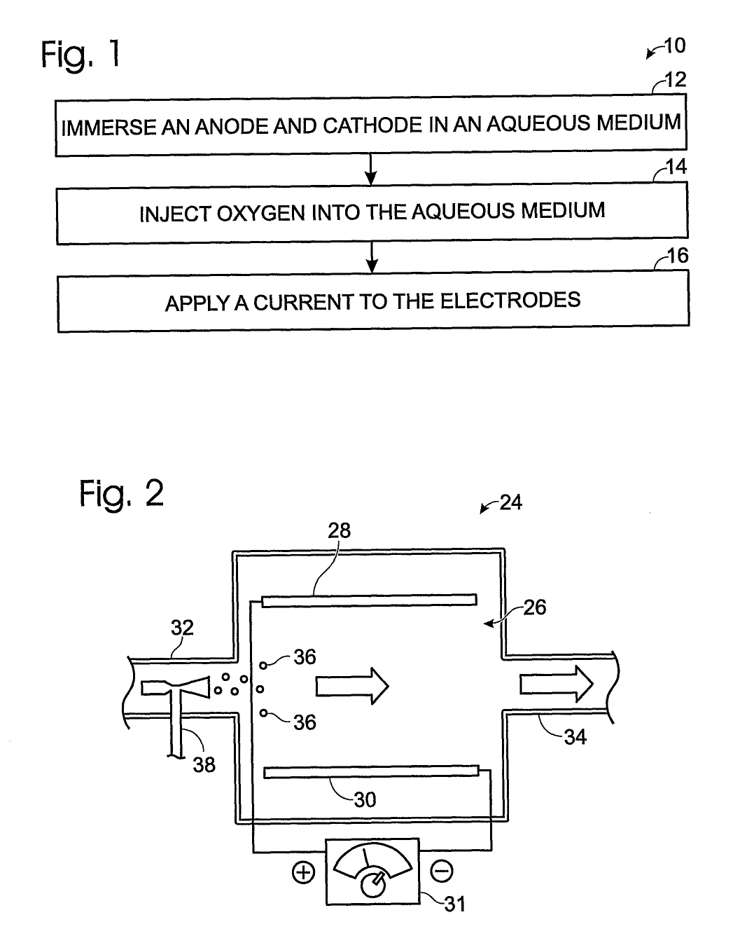

The preferred embodiments described herein include electrolytic methods for increasing oxygen concentrations in aqueous systems, as set out in flowchart 10 of FIG. 1. The oxygenation method typically includes substantially immersing an anode and a cathode in an aqueous medium 12, injecting oxygen into the aqueous medium 14, and applying a current to the electrodes 16.

As shown in FIG. 2, while the anode and cathode electrodes may be inserted directly into the aqueous medium, they are typically placed in an electrode cell 24 that also contains at least some of the medium to be treated 26. The anode 28 and cathode 30 are generally inserted into the medium sufficiently far so that they are substantially immersed in the medium, and current is applied to the electrodes by an associated power supply 31. Where the electrode pair is located in an electrode cell, the cell is optionally a flow-through cell having an intake 32 and an output 34, so that a flow of the aqueous medium can be configured to pass through the cell. Oxygen 36 is introduced to the aqueous medium via an oxygen injector 38.

The electrolytic oxygenation process typically generates oxygen levels in the aqueous medium greater than the saturation point for that medium, that is, the oxygenation process results in an aqueous medium that contains more dissolved oxygen than could normally be dissolved by that solvent under existing conditions of temperature and pressure. Such a medium is referred to as ‘supersaturated’, ‘oversaturated’, or ‘super-oxygenated’.

The electrolytic oxygenation process is useful in a variety of applications, including without limitation therapeutic uses, uses in aquaculture, and the purification of aqueous media such as wastestreams. The methods and processes described herein are generally applicable regardless of the particular electrode used or the particular chemical make-up of the aqueous solution, and are generally compatible with living systems, including freshwater aquatic lifeforms. The commercial viability of the instant oxygenation process is enhanced through the elimination of the need for added electrolyte, as many of these additives may create unwanted chemical species or reactions outside of the electrolytic process itself.

Oxygen gas may be added to the aqueous medium under treatment by any suitable method, including without limitation the injection of air, compressed air, gaseous or liquid oxygen, or other sources of oxygen. The oxygen may be injected at any point in the system undergoing treatment, but is typically injected prior to or within the electrode cell.

Any method of adding oxygen to the aqueous medium that results in increasing the oxygen concentration in the medium is a suitable method of adding oxygen. Any of a variety of aerators, bubblers, and injectors may be used to add oxygen to the aqueous medium. Typically, oxygen is injected using a venturi-type injector. In one aspect, oxygen is injected using a MAZZEI brand venturi-type injector.

Typically, the electrolytic treatment includes immersing a pair of electrodes (a cathode and an anode) in the medium and applying a potential, with a corresponding current output at the electrodes. The applied potential may be at least 10 volts, or more typically at least 20 volts. The oxygenation process may employ currents of greater than about 5 amperes, and more typically employs currents of greater than about 10 amperes. The use of electrode currents greater than about 20 amperes, greater than about 30 amperes, or even greater than about 40 amperes may also be advantageous in selected applications.

Typically, the applied DC voltage may be modulated to include a regular or irregular waveform superimposed on the DC potential. The applied waveform is typically a regular waveform, and is preferably a sine waveform. This sine wave ‘ripple’ is typically no more than 3% of the applied potential, and is preferably no more than 1.5% of the applied potential.

Where the electrodes are inserted into a flow of an aqueous medium, the flow may correspond to a variety of aqueous systems, including without limitation, therapeutic immersion media, aquaculture media, wastewater, or discharge from any of a variety of industrial processes. The aquatic media treated according to one of the present methods may be present as a static (non-circulating) supply, or the aquatic media may be circulated within a given volume, or recirculated from a reservoir or holding tank back to the electrode cell for additional treatment. The water treatment system may optionally include any of a variety of additional filters, pumps, holding tanks, settling tanks, additional media reservoirs, or other components known in the art. Although the oxygenation process may be sufficiently effective that the medium may be utilized after a single passage through the electrode cell, the electrode cell may optionally form part of a recirculating treatment system. After treatment, the treated aquatic media may be retained for use, retained for retreatment, or discharged.

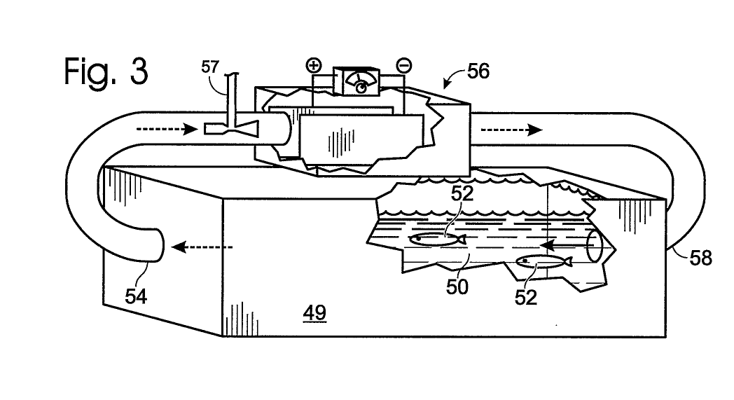

The electrolytic process may be used to treat a freshwater aquaculture medium, or the water supply used to support one or more stocks of cultured aquatic species, as shown schematically in FIG. 3. Aquaculture tank 49 may hold aquaculture medium 50 and one or more stock species 52. The aquaculture medium 50 may be pumped via water intake 54 to an electrode cell 56, where oxygen is injected into the electrode cell via injector 57 and the medium is electrolytically treated. The oxygenated medium is returned to tank 49 via discharge pipe 58.

The aqueous medium undergoing treatment may be a freshwater aqueous medium. As used herein, freshwater media typically have a salt content of less than about 0.5 parts per thousand (5,000 ppm).

In addition to the use of high applied currents, the use of overpotential voltage levels in the treatment process may also enhance oxygenation. Very high applied electrode potentials may effect the desired oxidative or reductive reactions used to treat the wastewater. This applied potential typically corresponds to between about 30 and about 100 volts DC. The utilization of such high electrode potentials typically corresponds to an “overpotential” in the electrolytic system under treatment.

Although a given overpotential value is dependent upon the electrode material and on the current density, generally speaking, the greater the rate of electron transfer desired, the greater the overpotential that should be applied.

Electrolysis of aqueous systems typically generates a variety of active oxidizing and reducing agents. Oxidizing agents typically produced during aqueous electrolytic treatment may include, without limitation, monotomic oxygen, singlet-state diatomic oxygen, hydroxyl radicals, hydrogen peroxide, and superoxide anion. In general, the greater the applied overpotential, the greater the amount of oxidizing agents produced during treatment, regardless of the particular solution pH or temperature.

However, the injection of even small amounts of oxygen gas into the media prior to electrolysis has been found to create high levels of oxy-radicals, including, but not limited to, polarized O₂ (O₂⁻ and O₂⁺). The use of ozone (O₃) gas did not perform or provide the same beneficial results, even when levels of ozone gas injection exceed that of oxygen gas (O₂) by a factor of 7:1.

Using a venturi-type injector, various rates of oxygen gas injection were evaluated, including 0.25, 0.50, 0.75, 1.0, 2.0, 3.0, 5.0, 6.0, and 7.0 liters per minute (Lpm), respectively, and the resulting concentration of dissolved oxygen was measured. No apparent gain was achieved where oxygen gas injection rates exceeded 7.0 Lpm per electrode chamber.

Electrodes. The particular physical configuration of the electrodes used in the oxygenation processes of the invention are typically not critical to the efficacy of the treatment. The electrodes used may take any of a variety of physical forms, including a mesh, a rod, a hollow cylinder, a plate, or multiple plates, among others. The electrode must typically provide sufficient surface area for creation of the necessary electrolytic field when oxygenation is conducted.

Although a particular electrode configuration is not required for performing the oxygenation process, the use of plate electrodes may be advantageous. In particular, plate electrodes having between 8 and 11 electrode plates that are spaced approximately 0.5 inches apart in parallel have been shown to permit the application of a significant overpotential in even low-conducting water streams (such as tap water). As long as the plate electrodes are configured so that the likelihood of capturing any solids from the media flow is minimized, the configuration of each plate is not critical. For example, the plate electrodes may be substantially solid or include a hexagonal mesh. By minimizing the possibility of fouling by organic or inorganic solids, the chances of forming a short between the anode and cathode are minimized, and typically the media flow restrictions through the electrode cell are simultaneously reduced.

The particular composition of the electrode may not be overly critical, provided that the electrode material is sufficiently robust to withstand the voltage and current levels applied during the electrolytic process, without excessive degradation of the electrode. A given electrode may be metallic or nonmetallic. Where the electrode is metallic, the electrode may include platinized titanium, among other compositions. Where the electrode is nonmetallic, the electrode may include graphitic carbon, or any of a variety of conductive ceramic materials. Ceramic electrodes have the potential of providing enhanced durability, biocompatibility, and affordability. It may be preferable that the electrode composition is selected so that metal oxides are not leached into the media, to the detriment of either aquaculture stock species, or the recipients of therapeutic treatment.

The anode and cathode of the electrode cell may have any of a variety of different compositions and/or configurations. The anode and cathode may also be substantially equivalent in order to facilitate bipolar operation, as discussed below.

The electrode cell used to carry out the electrolytic process of the invention may also include a reference electrode. A reference electrode is an electrode that has a well known and stable equilibrium electrode potential that is used as a reference point against which the potential of other electrodes may be measured. While any electrode that fulfills the above requirements is a suitable reference electrode for the purposes of the invention, typical reference electrodes include silver/silver-chloride electrodes, calomel electrodes, and normal hydrogen electrodes, among others.

Bipolar Operation. Electrolytic processes may generate thin films or deposits on the electrode surfaces that can lower the efficiency of the water treatment process. Descaling of the electrodes to remove some films may be carried out by periodically reversing the polarity of operation (switching the anode and cathode plates to the opposite polarity). Automatic logic controls may permit programmed or continuous descaling, further reducing labor and maintenance costs. Alternating electrode bipolar operation may increase the ability to continuously treat a given water stream, and decrease the rotational time required for effective oxygenation.

Voltages Rotation And Time. Electrolytic treatments of water may be dependent upon time and “rotation”, where rotation is the number of times that the medium under treatment has passed through an electrode chamber. The progress of a given course of water treatment may be measured as a function of the water rotation and the amount of voltage applied. As there are numerous possible chemical reactions and equilibria being created and destroyed simultaneously inside the electrode chamber, no set mathematical formula exists for predicting the number of rotations and voltage output required to oxidize a specific chemical compound or species. However, there are formulae that are applicable to recirculation in a closed system that may assist in determining the actual number of rotations necessary to treat a specific body of water. For example, formulae exist for determining the theoretical number of rotations of a known water volume through a given pump, filter, electrode chamber, etc., so that at least 99.9% of the water volume has passed at least once through the pump, filter, electrode chamber, etc. However, actual results must be based on previous test results or other experimentation in order to determine the best treatment regime for a particular water sample, as no two water systems contain the exact same pollutants and/or chemical compounds.

Catalytic Enzymes. “Catalytic enzymes,” as used herein, refer to enzymes that are useful in the degradation and/or solubilization of organic matter. Catalytic enzymes have been widely used to speed the oxidation of hydrocarbons and aromatics from fuel and crude oil spills in both marine and freshwater. Catalytic enzymes serve as a concentrated source of enzymes capable of catalytically accelerating the digestion of waste accumulations and aiding the elimination of organic accumulations in the water undergoing electrolytic treatment. Advantageously, Catalytic enzymes have also been found to aid in selected therapeutic treatments, as discussed below.

Useful catalytic enzymes include without limitation one or more members of the following enzyme classes: phosphatases (including alkaline phosphatase and acid phosphatase), esterases, catalases, dismutases, nucleotidases, proteases (including peptidases), amylases, lipases, uricases, gluconases, lactases, oxygenases, and cellulases. Preferably, the catalytic enzymes used in the present invention include one or more hydrolytic enzymes, or hydrolases. For example, a mixture of catalytic enzymes may include one or more protease enzymes, one or more amylase enzymes, and one or more lipase enzymes. The particular composition of enzymes used may vary with the type and amount of contaminants in the water undergoing treatment, and the amount and type of catalytic enzymes added may therefore be tailored to the individual situation.

Catalytic enzymes may be added to the medium undergoing treatment before or during electrolytic oxygenation. The catalytic enzymes may be added in substantially pure form, or added as a homogeneous or heterogeneous mixture that includes other components. A particular source of catalytic enzymes useful in conjunction with the treatment described herein is Orenda Technologies (Trumbull, Conn.), which supplies suitable enzyme mixtures under the product names CV-600, CV-605, CV-610, and CV-635.

An additional advantage of the use of catalytic enzymes is the reduction or elimination of biofilms at the electrode surface. A “biofilm” is the result of growth of various living organisms on the electrodes, and is common in fresh and marine water systems. Such microorganism growth increases scaling at the electrode surface, and reduces the efficiency of the electrode, requiring increasing voltage levels in order to yield the same results. The presence of active catalytic enzymes may dissolve biofilms already in place, and help prevent the formation of new biofilms. In particular, the use of catalytic enzymes in conjunction with periodic bipolar operation (as described above) may reduce or even eliminate routine electrode maintenance, which has been a commercially limiting factor in other electrolytic treatment processes.

Flocculating Agents. In some cases, a flocculating agent may be added to the water undergoing electrolytic oxygenation, to help clarify the water or selectively remove one or more impurities. Gentle mixing of the water and the flocculating agent typically causes the selected impurities or other particles to coagulate into larger floc particles. The larger floc particles may then be removed by sedimentation, filtration, or other processes. Selected flocculating agents include charged polymers (including cationic or anionic polymers), ferric chloride, aluminum sulfate (alum), and lanthanum (III) chloride, among others.

A flocculating agent may be added to the water undergoing treatment in combination with one or more catalytic enzymes, or other treatment additives. The catalytic enzyme mixture CV-635, as sold by Orenda Technologies (Trumbull, Conn.), already includes lanthanum (III) chloride.

### Applications

Aquaculture Media The electrolytic oxygenation process has been found to exhibit substantial utility for freshwater aquaculture. In particular, the oxygenation process is able to produce oxygen levels substantially higher than saturation levels, even at very high elevations, where it is typically very difficult to increase dissolved oxygen levels.

In particular, as demonstrated on a recirculating freshwater tilapia system, the injection of oxygen gas into the water stream prior to its entrance into the electrode cell provided 100% transference of the oxygen gas. In the absence of electrolysis, and using either micro bubble diffusion or liquid oxygen injection, substantially increased oxygen pressures would have been required to achieve the same level of oxygenation. The electrolytic oxygenation process permits small and inexpensive oxygen concentrators to be used for oxygenation, rather than expensive liquid oxygen, and that 100% transference of oxygen to the water can be achieved without use of high-pressure oxygen tanks.

The oxygenation process may be combined with electrolytic treatment of the aquaculture media in order to reduce ammonia, nitrite and nitrate levels, the main pollutants generated by the aquatic species as they are reared, resulting from the stock species metabolic process and respiration, fecel material and/or excess feed. Electrolytic oxygenation is also able to reduce the numbers of pathogenic bacteria in aquaculture media, including aeromonas, pseudomonas, septicemia, streptococcus, as well as various destructive molds, and fungi. This effect is enhanced where catalytic enzymes are also utilized. The use of electrolytic treatment in combination with catalytic enzymes has also been found to inhibit algae growth in aquaculture media.

The use of electrolytic oxygenation to treat aquaculture media may increase the commercial viability of freshwater aquaculture, particularly at higher elevations, or where oxygenation using conventional methods is economically unfeasible or technically impractical.

At an injection rate of 7 Lpm, an oxygen concentration of above 360% (supersaturated) was achieved. This level of supersaturation was achieved using at an injection rate of 6 Lpm at an altitude of more than 5,240 feet above sea level, and at a water temperatures of 90 degrees F. Additional tests at sea level with water at a temperature of over 152 degrees F. resulted in a level of supersaturation of 330% at an injection rate of as little as 3 Lpm. It was noted that the rate of silicate, fluoride, and mercury removal (see Examples 6, 7, and 8) were not increased significantly even when dissolved oxygen levels surpassed 360% saturation.

By using the oxygenation process described herein, the injection of relatively small amounts of 92-94% pure oxygen gas may result in an increased level of oxy-radicals and polarized oxygen molecules beyond levels that can be obtained via simple electrolysis of the aqueous solution alone. Additionally, the presence of oxygen gas (O₂) is more important then ozone O₃. Without wishing to be bound by theory, injection of oxygen gas may result in the creation of larger numbers of OH radicals and free hydrogen, than may be generated by cleavage of O₃ with the subsequent recombination of non-polarized O₂ with free hydrogen ions.

Therapeutic Applications

The oxygenation process described herein may be used in a variety of therapeutic applications, including applications with human subjects. By at least partially submerging test subjects in a reservoir of freshwater that has been or is being electrolytically oxygenated, a variety of health benefits have been observed. Without wishing to be bound be theory, it is believed that such treatments permit polarized oxygen to permeate the skin of the subjects, leading to an increase in blood oxygen levels. This increase may confer a variety of health benefits, as described below, including without limitation decreasing blood pressure in the aortic valve, as well as increasing blood flow to lesions associated with psoriasis.

Therapeutic treatments were conducted with water having dissolved oxygen levels of 194% to 280% of saturation. It was found that for predominately “healthy” individuals, oxygen saturation levels of 194-220% was more then sufficient to observe beneficial results. For those suffering diminished lung capacity or heart damage, increased levels of oxygen supersaturation was efficacious, for example oxygen levels of 250-280% were determined to be useful.

These levels of oxygen saturation were achieved at minimal amperage output at the electrode cell, typically less then 2.4 amperes, although this is more of a function of interelectrode spacing as no added electrolyte solution or electrolytic additives were used. In some cases, a potential of 20 V DC may be required to achieve and maintain these elevated oxygen saturation levels, for example in an open tub of freshwater at 103 degrees F. while the test subject was at least partially submerged.

None of the water used for the therapeutic treatments utilized an added sanitizing agent. The initial and all subsequent re-fill water is well-water, receiving no treatment with chlorine, bromine, UV-light, ozone, or other sanitizing agent.

A solution of approximately ¼ cup of distilled water containing 2 mL of a concentrated hydrolase enzyme formula (Orenda Technologies) was added on either a daily, or every other day basis to the tub water for all tests. Hydrolase enzymes are well documented in the medical industry for utility in dissolving necrotic tissue from human and animal wounds, without damaging living tissue. Additionally, the enzyme formulation utilized in these tests has been proven to solubilize hydrocarbon-based oils. It is believed that the use of the hydrolase enzymes breaks down the natural oils residing on the outside of the skin of the test subjects.

Without wishing to be bound by theory, solubilizing these skin oils allows for greater oxidation of yeast and bacterial infections, as described below, and for faster reduction of the necrotic tissue associated with scars and lesions. Although it has not previously been described, it is believed that the use of such enzymes may remove or reduce pre-existing scar tissues or lesions, and confer additional health benefits.

Over a period of several months, tests were conducted where additional enzymes were not added to the therapeutic immersion medium. Scar and/or lesion reduction still occurred. However, eczema patches did return to approximately 20% of coverage of the area previously exposed to the treated water and enzyme mixture.

The following examples are included for illustration and are not intended to limit or define the entire scope of the invention.

### EXAMPLES

### Example 1

### Oxygenation Treatment of a 62-Year Old Female with a Damaged Aortic Valve

The subject has a damaged aortic valve resulting from the use of the dietary drug combination fen-phen. The subject suffers from PPH—primary pulmonary hypertension and aortic stynosis, and has been administered 3 Lpm of pure oxygen gas, 24 hours a day for over 5 years. The oxygen gas flow required to maintain her blood oxygen levels at 93-94% were exceeding 3.5 Lpm, which is the maximum allowed without incurring serious damage to nasal mucus membranes and lung tissue. The subject additionally has suffered from psoriasis for several years, continuously covering an area from the ankle to the lower back, due to the constant injection of the medication Flolin by direct injection into her lungs, permitting the lungs to transfer oxygen to the blood. Additionally, the subject requires 1-2 tablets 0.2 mg nitroglycerin per day to reduce the occurrence of aortic stenosis (enlarged aortic valve). In aortic stenosis, the aortic valve is weakened, requiring the need for nitroglycerin to both thin the blood and reduce blood pressure.

Prior to her exposure to an electrolytically oxygenated bath, the subject had been denied heart surgery to repair and/or replace the damaged aortic valve, as it was the determination of several heart specialists that the subject would not survive the operation. She had been advised to begin seeking hospice care. Within 5 days of the exposure to the oxygenated water each day for 30-40 minutes, her need for nitroglycerin decreased by over 50% per day on average, with no nitroglycerine required on numerous days, depending on her activity level.

The woman was exposed to oxygenation treatment using a 150-gallon poly tank containing water heated to 102 degrees F. and having with a dissolved oxygen level exceeding 280%. The subject was treated for 30 minutes each day for 10 days. During the first 3 days of treatment, the woman's oxygen level rose to 98% without direct oxygen gas while in the tank, and remained at that level for approximately 60 minutes after exposure. Within 7 days of the treatment, the woman was able to maintain her blood oxygen level at 95-96% for 5-6 hours following treatment, and reduce her oxygen feed to 2.5 Lpm thereafter. After the 10th day, the woman was able to maintain her blood oxygen level for as long as 8 hours, and required only 2 Lpm of direct oxygen gas thereafter.

The electrolytic oxygenation treatment was suspended for two days (days 11 and 12) though the woman still spent 30-45 minutes in the heated water per day. No increase in blood oxygen levels were achieved when the oxygenation treatment was halted, and the woman had to maintain 2 Lpm of direct oxygen feed the entire day.

A further delay of 5 days was initiated, stopping even the soaking in the hot water tank. The woman's blood oxygen level fell by the 5th day back to 93% with 3.5 Lpm of direct oxygen feed.

Exposure to the electrolytic oxygenation process was again performed, with the dissolved oxygen level of the water reduced to less then 200% saturation. The individual's blood oxygen level again rose to 98% and she was able to reduce her required direct oxygen feed over the next 48 hours from 3.5 Lpm to 2.5 Lpm, where it remained thereafter.

A medical evaluation of the subject's aortic valve was performed 5 days later, in a determination as to whether the blood pressure within the aortic valve would permit surgery on the valve. The ultrasound examination of the valve determined that the pressure had reduced over 50% compared to the pressure measured during an evaluation 6 months previously.

### Example 2

### Oxygenation Treatment of Psoriasis

As indicated in Example 1, the subject of that example also suffered from psoriasis from her ankles, up her legs to her lower back region. Typical of psoriasis, her body had formed exterior lesions where the epidermis was 7-8 times thicker then normal. A T-cell reaction that is further exacerbated by yeast and bacterial infections created a severe rash accompanied by itching. Typical treatment of this condition is with topical analgesics, but since yeast infections may result from heavy antibiotic use, there is no other topical treatment that is normally effective. The application of oxidants such as hydrogen peroxide is typically extremely painful.

During the oxygenation treatment described in Example 1, it was noted that the rash created by the psoriasis was reduced in area by over 85%, accompanied by a visible reduction in the raw-reddish appearance at the ankle area, becoming a light pink color. There was a concomittant reduction in itching, reducing her need to apply a topical immunosuppressant (ELODEL) to only once every few days, instead of several times per day as previously required.

The subjects lesions became noticeably more pliant after treatment, and the lesion color went from looking like bleached scar tissue, to a healthy pink. It has been previously noted that the electrolytic creation of oxy-radicals may inactivate pathogenic bacteria, viruses, mold, and fungus. However, no previous description of the treatment of yeasts in this manner has been made, particularly yeasts inhabiting the exterior of the human epidermis. It appears that the electrolytic oxygenation treatment creates sufficient oxy-radicals, as well as polarized O₂ (polarized O₂ is classified as an oxy-radical ion species) to inactivate yeast.

During those periods where the electrolytic oxygenation treatment was not performed, either by soaking in the heating tank without oxygenation, or by not soaking at all, the yeast infection appeared to return, and the more severely affected areas began returning to a rawer, blistered state.

One existing treatment for psoriasis involves the use of a hyperbaric chamber, with both oxygen and oxygen-ozone mixed gases being used to reduce external yeast infections, and to supersaturate the patient's blood with oxygen. Unfortunately, hyperbaric chambers cannot be used by infants, many elderly individuals, or those suffering heart and/or lung damage.

Immersion in an aqueous medium with electrolytic oxygenation may be used to successfully treat those patients suffering psoriasis that are otherwise unable to receive hyperbaric chamber treatment, and result in the same, or greater, reduction of symptoms.

### Example 3

### Reduction of Scarring and Lesions

The subject of Example 1 also exhibited numerous psoriatic-based lesions. After 4 weeks of daily exposure to electrolytically oxygenated water, at dissolved oxygen levels of 220% or greater saturation, the lesions were reduced in size by over 95%, with over 90% of the lesions completely being replaced by new skin, without scarring or indication that the lesions had been present.

A second woman, 50 years of age, with an ethnicity that included 25% Choctaw Indian ancestry, had several keloid-type scars on her thighs. The scars were the result of injuries in her early teens, and the general size of the scars was approximately ¼″ in diameter, raised above the surrounding epidermis by ⅛″. After 10 days of electrolytic oxygenation treatment, the scars were reduced in size and height by 50%.

Additionally, the subject had a chemical-burn scar approximately 2″ in diameter located on her upper front left hip. The scar is discolored and raised in appearance. Again, after 10 days of treatment, the scar was noticeably lighter in color, with much of the interior epidermis showing a healthy pink color. The scar was also noticeably smoother and shallower in appearance.

This subject had also suffered from shingles (Herpes Zoster) for over 7 years. Typical of shingles, each “outbreak” would consists of the forming of raised blisters, accompanied by severe pain as well as a burning sensation in the affected area. In particular, this subject experienced shooting pains in her legs and lower back. After beginning electrolytic oxygenation treatment, the blisters became smaller, and the blisters did not form a scab. The “outbreak” period, the time from the beginning of the pain to the point where the blister forms, has become shorter, and the symptoms are not as severe. On a scale of 1-100%, the duration time is 25% shorter, with the pain reduction being approximately 10-15%.

The skin in the area where the outbreak and blistering routinely occur has become discolored from the numerous blister formations. It has been noted that after treatment, the discolorations are 95% gone, with the new epidermis showing no signs of discoloration or scarring.

A male, age 62 years who is 80% bald, exhibits age-related melanin spots on his face, forehead, and top of his head where bald. These age spots have been present for at least 9 years. The subject treated the age spots by splashing his face and forehead with treated water, and with 5-minute “soaking” of the top of his head by reclining backwards in the tub. The electrolytic oxygenation process resulted in dissolved oxygen levels of 290% at 100 feet above sea level at a water temperature of 103 degrees F. Following 14 days of treatment, the subject exhibited a 60-65% reduction in the discoloration spots.

Another male subject, age 45 years, exhibited both age spots on his hands and forearms as well as a benign scab growth that was constantly present on the top of his head, where he was bald. This growth had been diagnosed as a melanoma-type growth, probably due to many years of over-exposure to the sun. The area on his head had been consistently sore to the touch for over 5 years, with a pattern of scab-like growths being present at two to three different points fairly continuously over that time.

The subject was treated over 10 days using a hot tub containing treated water having 208% dissolved oxygen at 103 degrees F. After treatment, the age spots on the backs of his hands and forearms were 99% gone. The subject also reported that within 2-days of 3-5 minute exposure of the scabbed area of his head to the treated hot tub water the scabs were gone and there was no soreness to the area. Over the next 10-14 days, he noted that the scabs did not reappear, except for one, very small point (with an area of about 2 mm) that appeared and disappeared, but that the soreness has not returned.

The 62-year old male discussed above has had a raised, eczema-like growth behind his left ear, covering an area about the size of his thumb, for several years. After his first 14 days of treatment with approximately 5-15 minute exposures to the electrolytically oxygenated hot tub water, the growth was reduced to an area of 2-3 mm.

The 62-year old male and the 50-year old woman previously noted have both experienced the loss of various moles located on the arms and back after treatment. The area where the moles had been was unmarred and no residual indication that the moles were ever present is apparent.

### Example 5

### Treatment of Fungal Infections

The 62 year-old male subject previously noted has suffered from fungal growth beneath his large toes for over 5 years. Topical agents and lotions have provided a modicum of temporary relief, but have not been able to cure the fungus permanently. The fungus created a large mass of yellow dead-looking skin under the toenail, so that the toenail was raised and discolored.

After two 30-minute exposures to the treated water, the size of the fungal growth was noticeably reduced. Over a period of 10 days, the growth receded to the point where the void under the toe nail was almost 100% larger. Since the subject began daily soakings of 20-30 minute duration in the treated water, the fungus has not returned and the toenail has begun to return to its normal position on top of the toes epidermal layer.

### Example 6

### Water Treatment

Treatment of municipal drinking water and reclaim water was undertaken to reduce silica and heavy metals prior to introduction to membrane filtration for use in steam boiler electrical generation. A bench test of the water was made with the electrolytic oxygenation process, followed by filtration by 3-nominal bag filtration, DE (diatomaceous earth), and mixed cationic/anionic media.

Dual tests were performed. Test #1 was performed with only ambient air injection through a 1.5″ MAZZEI brand air injector. Test #2 was performed with the use of the MAZZEI brand air injector, but with a pure oxygen gas feed of 3 Lpm. The same test water, as well as the same operating parameters were used in each test.

Test #1 produced a 45% reduction in soluble silica. Test #2 produced a 99.5% reduction of soluble silica under the same applied amperage to the electrode.

### Example 7

### Silica Removal

Both colloidal and soluble silica in water create severe fouling in membrane filtration, as well as scaling in cooling towers and steam generation electrical boilers. Bench tests on geothermal well water at 156 degrees F. as well as 48 degrees F., and on municipal Reclaim wastewater at 42 degrees F., indicate that where dissolved oxygen levels exceed 150% and applied current levels exceed 30 amperes, that both colloidal and soluble silica compounds are oxidized and become insoluble precipitates, regardless of water temperature and pH. Tests indicated that at higher amperages, those exceeding 59 amperes, silica compounds are removed efficiently, even where silica levels exceeded 129 mg/L.

Silica reduction follows fluoride reductions in water, and has been determined to be both oxygen concentration- and applied current-dependent. Applications of current less then 40 amperes, with dissolved oxygen levels less then 100% will not efficiently remove fluoride. However, the application of amperage exceeding 40 amperes and dissolved oxygen levels over 126% of saturation results in the precipitation of fluoride from the water column following exposure to the electrolytic field.

### Example 8

### Mercury Removal

Mercury is a pollutant found in many subsurface aquifers. Several tests on both municipal reclaimed wastewater and industrial wastewater from aluminum manufacturing processes have shown significant mercury reduction where dissolved oxygen levels were raised to over 200%, regardless of the water temperature or pH. The mercury was found to have come out of solution, requiring as little as 3-micron filtration to capture the precipitate and remove it from the solution.

Where applied voltage and amperage levels exceeded 40 volts/40 amperes, and oxygen gas was not fed into the water stream prior to the electrode chamber, mercury reduction was not noted. However, once as little as 3 Lpm of 92% pure O₂ gas was injected into the water stream ahead of the electrode chamber, and the same amperage was applied, mercury was removed from the water column.

Additional tests on more conductive water showed that voltage could be reduced to less then 20 volts if a current level of greater than 50 amperes was maintained, and dissolved oxygen saturation levels remained at over 126%. Saturation levels were found to be tied to applied voltage, even where suspended solids levels were recorded at over 2000 mg/L.

### Example 9

### Oxygen Injection in the Absence and Presence of an Applied Electrolytic Field

Small amounts of O₂ gas injected prior to the electrode flow chamber can provide a 10-fold increase in oxygen super-saturation levels, a degree of oxygen saturation greater then can be achieved by injection the same amount of oxygen via fine bubbler aeration or via MAZZEI brand venturi injector infusion (depending on voltage levels applied). The ability to reach such levels regardless pH level and temperature provides wastewater treatment operators with a very cost effective tool to increase treatment capacity and reduction rates.

Oxygen is injected into a water stream flowing at a rate of 35 gpm (gallons/minute), into 70,000-gallons of freshwater at 62 degrees F. at 700′ above sea level.

| | |
|---|---|
| Dissolved oxygen (DO) without O₂ injection: | 68% |
| DO with O₂ injection using MAZZEI brand injector after 3 days: | 84% |
| DO with O₂ injection and electrolytic treatment after 36 hours: | 126% |

The effect of electrolytic oxygenation was measured on 55,000 gallons of tilapia-rearing water at an altitude of 5,240′ above sea level. The aquaculture medium was supporting 40,000 lbs of fish, with a water temperature of 82 degrees F.

| | |
|---|---|
| DO with fine bubble aeration and electrolysis alone | 30% |
| DO with 15 Lpm O₂ aeration through fine bubble diffusers: | 38% |
| DO with 15 Lpm O₂ injection using MAZZEI brand injector: | 48% |
| DO with 15 Lpm O₂ injection into electrode chamber | 116% |
| using MAZZEI brand injector: | |

The effect of electrolytic oxygenation was measured on 32,000 gallons of tilapia-rearing water at an altitude of 5,240′ above sea level. The aquaculture medium was supporting 10,000 lbs of fish, with a water temperature of 82 degrees F.

| | |
|---|---|
| DO with O₂ aeration through fine bubble diffusers (15 Lpm): | 65% |
| DO with O₂ aeration through electrode chamber (15 Lpm): | 285% |

The effect of electrolytic oxygenation was measured on 30 gallons of oil barge wastewater.

| | |
|---|---|
| DO with electrolytic exposure only: | 83% |
| DO with 3 Lpm of O₂ injected into electrode chamber: | 396% |

The effect of electrolytic oxygenation was measured on 55 gallons of aqueous fruit processing effluent, having a corn sugar level of 36-brix (36% sugar), with a water temperature of 152 degrees F.

| | |
|---|---|
| DO with electrolytic exposure only: | 56% |
| DO with 3 Lpm O₂ injected into the electrode chamber: | 287% |

The effect of electrolytic oxygenation was measured on industrial plastics manufacturing wastewater at a pH level of 1.53, and containing high levels (over 1000 ppm) of copper in solution, at ambient air temperatures:

| | |
|---|---|
| DO with MAZZEI injection of air and electrolytic action only: | 87% |
| DO with 3 Lpm O₂ injected via MAZZEI injector without | 74% |
| electrolysis: | |
| DO with 3 Lpm O₂ injected via MAZZEI injector with | 296% |
| electrolytic action: | |

The effect of electrolytic oxygenation was measured on 30 gallons of geothermal well water at 132 degrees F.

| | |
|---|---|
| DO with electrolytic action only: | 32% |
| DO with 8 Lpm O₂ injection only: | 39% |
| DO with 8 Lpm O₂ injection and electrolytic action: | 313% |

### Example 9

### Coagulation of Emulsified Petroleum by Electrolytic Oxygenation

The electrolytic oxygenation process may be used to effectively reduce total hydrocarbons (THC) and aromatics in aqueous wastewater, to levels that permit municipal treatment or direct-sea discharge.

Even after treatment by heat coagulation and oil-water separation by diffused air filtration (DAF), some wastewater streams may still include large amounts of residual emulsified oils (for example, greater than 10,000 mg/L). In order to satisfy selected environmental regulations, wastewater THC levels should be reduced to less then 30 mg/L and Aromatics below 5 mg/L.

The electrolytic oxygenation process reduces petroleum levels to less then about 5 mg/L THC and less then 1 mg/L Aromatics. It is believed that the oxygenation process first produces a super-coagulate of emulsified oils that then float to the top of the wastewater and may be removed by skimming. The oil component recovered by skimming may be sufficiently dewatered that it suitable for resale and/or further refining.

A container holding 270 US gallons of barge washout wastewater was treated with 30 minutes of agitation with injection of 70 gpm of 25 psi air using a 1.5″ MAZZEI-brand air injector. This oxygen treatment, without electrolytic action, resulted in a very thin, fine level of coagulated oil, but failed to remove 99% of the oil suspended within the water volume.

The wastewater was then treated for 30 minutes with 15 Lpm flow of 92% pure oxygen from an oxygen concentrator. While this resulted in a higher dissolved oxygen content of the wastewater, it did not significantly improve the coagulation of the emulsified oils.

The wastewater was then treated with 15 Lpm of 92% pure oxygen concomitant with the application of 30 Volts DC to the electrodes in the flow chamber, with a resulting current of 31 amperes. Within 15-20 minutes, a very thick and dense layer of coagulated oil was produced at the top of the water volume. the coagulated was readily and quickly skimmed from the surface of the wastewater. The overall appearance of the wastewater changed as oil was removed, and become significantly lighter in color, first a light brown, and within 45 minutes, a medium yellow color. At that point, the coagulation endpoint appeared to have been reached.

Subsequent lab analysis of the wastewater indicated a 66% reduction of THC and Aromatics due to the super-coagulation of the emulsified oil and removal by skimmer during electrolytic oxygenation.

After 4 hours of treatment of a similar wastewater sample without removal of the coagulated oil by skimming, testing showed that the wastewater below the coagulated oil at the surface exhibited a 95% reduction of THC and greater then 99% reduction in the aromatics.

### Example 10

### Oxidation of Glycols

Samples of storm water run-off contaminated with high levels of propylene, diethylene, and triethylene glycols from a natural gas pipeline transfer station, and samples of gas pipeline condensate containing monoethylene glycols, were treated by electrolytic oxygenation.

At an applied voltage exceeding 30 Volts and at an oxygen gas injection rate greater than 15 Lpm of 90-92% pure oxygen gas, each sample exhibited dissolved oxygen levels that exceeded 250%. This degree of supersaturation consistently raised the oxygen reduction potential (ORP) from untreated levels greater then −500 mV to levels exceeding +790 mV within minutes, resulting in oxidation of glycols to CO₂ and water vapor. The resulting water was permitted to be discharged according to EPA regulations (less than 0.01 mg/L for discharge to land and sea). The ability to create such rapid increase in ORP permits the economical treatment of contaminated water volumes ranging from 20,000 to 2,000,000 gallons per day.

### Example 11

### Oxygenation of Oil Drill Cuttings

The treatment of drill cuttings from oil and gas exploration is of major interest issue throughout the world. Presently, drill cuttings are treated by mechanical filtration, then barged to shore for further treatment. As the volume of such cuttings can be several thousand metric tons, the cost of barging the drillings is significant.

Tests were performed on limestone/dolomite based drill cuttings from the North Sea. Electrolytic oxygenation at applied amperages exceeding 59 amperes showed that the oil-containing shale could be fractured—exposing the oil (expressed as THC— total hydrocarbons and aromatics) to the oxy-radicals created in the water stream, and thus reducing the THC and aromatics to levels permitting direct discharge to the sea. Where oxygen gas was injected at a minimum rate of 15 Lpm of 90-92% gas to the water stream prior to exposure to the electrolytic field, the rate of THC and Aromatic reductions was significantly enhanced when released to the liquor—thus allowing for not only the reduction of oil in the shale/drill cuttings, but maintaining a low level of THC and aromatics present in the water/liquor at the same time. The economic impact of this ability is significant, as typical barging costs for drill cuttings per oil platform alone can exceed $25,000,000 US.

### Example 12

### Electrolytic Oxidation of Aromatic Hydrocarbons

Tests were performed electrolytically oxidizing wastewater containing chlorobenzene from pesticide and herbicide manufacturing. Chlorobenzene was rapidly oxidized, reducing as much as 99.4% of the Chlorobenzene from the wastewater.

Tests were then conducted to measure the oxidation of benzene, toluene, ethylbenzene, and xylene (commonly referred to as BTEX) in the condensate from natural gas pipelines. As a result, benzene was reduced by 94.09%, toluene was reduced by 95.28%, ethylbenzene was reduced by 100%, and xylene was reduced by 75.0%. The oxidation process was repeated several times with different condensate having varying levels of these pollutants, with the result that the electrolytic oxidation process successfully removes aromatic hydrocarbons.

### Example 13

### Creation of Stabilized Dissolved Oxygen Levels

Numerous tests of the disclosed electrolytic oxygenation process in freshwater bath tubs, and spas demonstrate that supersaturation levels of the dissolved oxygen level of the water can remain fairly stable, up to 80 hours after exposure to the process.

Tests at 80° F. demonstrate retained dissolved oxygen levels exceeding 220% saturation. Tests at 103° F. demonstrate retained dissolved oxygen levels at over 140% saturation. The tests were performed at approximately 300 feet above sea level.

Accepted chemical theory is that above 77° F. at sea level, water will not hold more then 100% saturation of dissolved oxygen, with levels below 100% being determined by temperature.

It can be a misapplication of the terminology in the literature to use “supersaturation” to describe dissolved oxygen levels within an aqueous medium, as supersaturation typically refers to gas bubbles within the medium. Super-oxygenation is perhaps a more appropriate term, as this describes various forms of dissolved oxygen at the molecular level, as opposed to gas bubbles.

Although it has been determined that the disclosed oxygenation process, including electrolytic application of DC voltage at levels exceeding 10 volts and applied current exceeding 0.5 amps with the injection of air or oxygen gas, produces increased dissolved oxygen, the resulting changes to the oxygen ions is unknown. Without wishing to be bound by theory, it is the view of the inventors that during the disclosed process a polarization of the oxygen ion has occurred, creating what is referred to in isoelectronchemistry as “gas magnecules” or in layman's' terms—clusters of oxygen molecules, as described by Santilli (Foundations of Hadronic Chemistry with Applications to New Clean Energies and Fuels, Kluwer Academic Publisher, Dordrecht-Boston-London, ISBN 1-4020-0087-1; hereby incorporated by reference).

Even when considering the combination of 100% transference of the injected oxygen gas with the established level of water molecule fracturing by the electrolytic process, accepted chemical theory cannot account for the levels of super-oxygenation reached using the disclosed method. The prolonged high oxygen levels in the aqueous stream point to the creation of new chemical species as described by Santilli.

Additional tests performed in South Africa in early 2006 by outside researchers utilizing the disclosed process give credence to this determination, as they demonstrated that the level of super-oxygenation achieved in 1000 liters of freshwater containing 100 pounds of live tilapia, at an altitude of 2,500 above sea level was over 400% of saturation. The level of oxygen gas plus the level of electrolytic fracturing of the water cannot account for this level of sustained saturation if only single oxygen molecules were present. However, clusters of oxygen with increased electrons could account for these reported levels.

### Example 14

### Magnecule Creation in Gases and Liquids by Application of DC voltage

Magnecules may be created by changing the polarity of the valence electrons in a plane, which signifies the change of the electrons from a spherical orbit, to one of a toroidal, and allows for the magnetic attraction and stable bonding of numerous molecules, whether of the same chemical or not. This has been accomplished using a plasma arcing process (PlasmaArcFlow™), which involves arcing AC electrical current through an aqueous media, creating plasma at approximately 5,000° C. of mostly ionized hydrogen, oxygen, carbon and other elements, which then combine in a variety of ways to form nonexplosive combustible gases. These gases, when burned, release no or minimal pollutants and have thus been labeled as “clean emission gases”.

As currently utilized, this process is highly energy intensive and expensive, as the equipment must be able to withstand the constant arcing of the electrical current through the aqueous media, and the gases must be rapidly cooled using cryogenic technologies in order to capture them.

Notwithstanding the need for rapid cold stabilization of the gases, the application of a properly configured wave of direct current, at voltages exceeding 10 volts DC and applied current sufficient to drive the chemical reaction, may be used to create gaseous and liquid magnecules.

In order to optimize this process, the sine wave of the output DC voltage should be configured within a specific range (referred in the electromechanical industry as the “ripple”). Based on the work of Santilli and others involved in the plasma arcing industry, and the results of the testing described herein, a particularly advantageous sine wave configuration when utilizing DC voltage can be formulated.

During reactions such as the oxidation of hydrocarbons, or the 100% transference of oxygen gas to an aqueous solution, applied kinetic energy (voltage) is of far greater importance then thermodynamic energy (current). This has been demonstrated in numerous tests. However, not all reactions are singly dependent on either kinetic or thermodynamic energy—some require both, such as the precipitation of fluoride, or the creation of magnecules. Typically, the output DC voltage ripple is no more then 3%, and preferably it is less then 1.5%.

It is noted that both hydrogen and oxygen magnecules as described by Santilli, have wide application in power creation industries, due to both the lower or non-emissions of the combusted gases, as well as the increased kilojoules of energy released per molecule. Essentially, Santilli and others denoted molecular weights from 4-6 times greater than H₂ and O₂. This increase in molecular weight could signify a greater energy release per molecule combusted in applications where hydrogen and/or oxygen are used as fuel sources.

The creation of hydrogen gas using electrolysis is well-known, and a common by-product of the creation of hydrochloric acid and sodium hydroxide from sea water. However, those technologies utilize course wave DC and/or AC voltage. By “course wave”, the inventor is referring to the sine wave of the electrical current, which in standard electrolysis applications has a wave form comprised of high peaks and deep valleys. This is because the processes denoted requires high thermodynamic energy (current) to create the desired chemical reaction, as compared to kinetic energy (voltage).

Regardless of whether the electromagnetic field created by the application of the DC voltage involves a substantially submerged electrode within an aqueous solution, or the aqueous solution is effectively injected into the electrode reaction chamber as a fine mist in an amount sufficient to achieve the required current across the electrode plates, the magnecules will be created where sufficient DC voltage and current within the aforementioned ripple, is supplied.

It is far less costly from both an equipment manufacturing and operational standpoint, to utilize DC voltage for the creation of magnecules then to utilize AC voltage. Additionally, it is far simpler and less costly to super-oxygenate an aqueous liquid using the presently disclosed process than to utilize liquid or gaseous oxygen and high pressure pumps and vessels. This process is therefore of great potential importance to firms, agencies, entities involved in water and/or wastewater treatment, and fuel production industries.

### Example 15

### Creation of Chlorine Dioxide

Chlorine Dioxide (ClO₂) is widely gaining acceptance as a preferred sanitizing agent over chlorine, chloramine, and bromide in virtually all water types, due to its longevity and its reduced creation of non-desirable by-products of oxidized organics.

Typically chlorine dioxide is prepared either through hazardous chemical reactions utilizing strong acids and reducing agents. Alternatively, chlorine dioxide can be produced electrolytically, however the traditional electrolytic process requires highly dangerous chlorine gas and the use of pressure vessels.

Tests utilizing the present electrolytic oxygenation process by the United States Department of Agriculture-Agricultural Research Station in Corvallis, Oreg., has determined that in the presence of a chloride source in a freshwater aqueous solution, where oxygen gas is injected by way of a water-operated venture injector, and sufficient DC voltage and current is applied with the prerequisite correct sine wave formation, chlorine dioxide can be created without the use of pressure vessels or chlorine gas injection.

The level of chlorine dioxide creation is adjustable by the operator, and determined by the level of both chlorides in the water stream and the amount of oxygen gas injected. Time of exposure to the electrolytic field (regardless of whether the electrode chamber and treatment regime is closed loop/batch or flow-through), chloride level, and amount of oxygen (gas or liquid) injected are all variables that allow the operator to adjust the process to maintain a desired level of chlorine dioxide within the water stream.

Although the present invention has been shown and described with reference to the foregoing operational principles and preferred embodiments, it will be apparent to those skilled in the art that various changes in form and detail may be made without departing from the spirit and scope of the invention. The present invention is intended to embrace all such alternatives, modifications and variances, including but not limited to those set out in the following claims:

What is claimed is:

1. An electrolytic method for oxygenating an aqueous medium, comprising:
substantially immersing an anode and a cathode in an aqueous medium;
injecting oxygen into the aqueous medium; and
applying a current to the electrodes.

2. The method of claim 1, comprising injecting oxygen into an electrode cell containing the anode and cathode.

3. The method of claim 1, comprising injecting oxygen prior to an electrode cell containing the anode and cathode.

4. The method of claim 1, employing a current of at least 20 amperes at the electrodes.

5. The method of claim 1, wherein the aqueous medium is supersaturated with oxygen thereby.

6. The method of claim 1, wherein a potential of at least about 10 volts is applied across the electrodes, and 100% of the injected oxygen is dissolved in the aqueous medium

7. The method of claim 6, wherein oxygen is injected without pressurization tanks or high-pressure micro bubble diffusers.

8. An electrolytic method for oxygenating an aqueous aquaculture medium, comprising:
substantially immersing an anode and a cathode in an aqueous medium;
injecting oxygen into the aqueous medium; and
applying a current to the electrodes such that the aqueous medium is supersaturated with oxygen.

9. The method of claim 8, wherein the anode and cathode are immersed within a flow-through electrode cell; further comprising directing a stream of the aqueous medium through the electrode cell.

10. The method of claim 8, wherein the aqueous medium supports one or more stock species.

11. An electrolytic method for treating water, comprising:
providing an electrode cell including a cathodic electrode and an anodic electrode;
directing a stream of water through the electrode cell, so that the electrodes are substantially immersed in the water stream;
injecting oxygen into the water stream; and
applying a current to the electrodes;
such that at least one pollutant is removed from the water stream.

12. The method of claim 11, wherein the pollutant is silica, mercury, or fluoride.

13. The method of claim 11, wherein the applied current is greater than about 40 amperes, and mercury is removed from the water stream by forming of an insoluble precipitate.

14. The method of claim 13, wherein the precipitate is removed by filtration.

15. The method of claim 11, wherein the applied current is greater than about 40 amperes and the method results in oxygen levels in the water stream of greater than about 126% of saturation, such that soluble and colloidal silicates are removed as a precipitate from the water stream.

16. The method of claim 11, wherein the pollutant is a petroleum contaminant.

17. The method of claim 11, wherein the pollutant is an organic contaminant.

18. The method of claim 17, wherein the organic contaminant is a glycol.

19. The method of claim 17, wherein the pollutant is removed from the water stream by coagulation.

20. A method of therapeutic immersion, comprising
substantially immersing an anode and a cathode in an aqueous treatment medium;
injecting oxygen into the aqueous medium;
applying a current to the electrodes such that the aqueous medium becomes supersaturated with oxygen; and
immersing at least a portion of a subject in the treatment medium.

21. The method of claim 20, where the subject exhibits an epidermal infection.

22. The method of claim 21, wherein the epidermal infection is a yeast infection or a bacterial infection.

23. The method of claim 20, where oxygen is injected into the treatment medium prior to exposure to the electrodes.

24. The method of claim 20, where the voltage applied across the electrodes is greater than about 20 volts.

25. The method of claim 20, where a blood oxygen level of the subject is increased by immersion in the treatment medium.

26. The method of claim 20, wherein the treatment medium has a dissolved oxygen content greater than about 220% of saturation.

27. The method of claim 26, wherein immersion in the treatment medium reduces the size of keloid and chemical scar tissue, lesions resulting from psoriasis, melanin-related age spots, benign melanoma, and eczema-related spots and lesions.

28. The method of claim 26, wherein immersion in the treatment medium reduces the size of exterior moles.

29. The method of claim 20, further comprising adding catalytic enzymes to the treatment medium

30. The method of any of claims 1, 8, 11, and 20, wherein the current is applied at a potential having an imposed sine waveform with an amplitude of about 3% or less of the amplitude of the applied potential.

31. The method of any of claims 1, 8, 11, and 20, wherein the current is applied at a potential having an imposed sine waveform with an amplitude of about 1.5% or less of the amplitude of the applied potential.
